(19) United States
(12) Patent Application Publication
(10) Pub. No.: US 2024/0018483 A1
(43) Pub. Date: Jan. 18, 2024

(54) A DEVICE AND METHOD FOR VASCULARISING A CELL AGGREGATE

(71) Applicant: AGENCY FOR SCIENCE, TECHNOLOGY AND RESEARCH, Singapore (SG)

(72) Inventors: Giulia ADRIANI, Singapore (SG); Andrea PAVESI, Singapore (SG)

(21) Appl. No.: 18/255,146

(22) PCT Filed: Dec. 1, 2021
(86) PCT No.: PCT/SG2021/050743

§ 371 Date: May 31, 2023

(30) Foreign Application Priority Data
Dec. 1, 2020 (SG) 10202011995P

(51) Int. Cl. C12N 5/071

(52) U.S. Cl. CPC C12N 5/0691; C12N 2513/00; C12N 2527/00

(57) ABSTRACT

There is provided a device for vascularising a cell aggregate, the device comprising: a matrix region configured to contain a gel-like matrix and cells that form a vasculature network within th ematrix region. The matrix region having at least one opening for positioning the cell aggregate therein based on a desired three-dimensional spatial location; and one or more fluidic regions configured to contain a supporting fluid that is capable of supporting vascularisation of the cell aggregate, the one or more fluidic regions being in fluid communication with the matrix region, wherein a flow passage from the one or more fluidic regions to a gel-like matrix disposed in the matrix region is configured to allow three-dimensional vascularisation around the cell aggregate and perfusion of the vasculature once formed. There is also provided a chip comprising a plurality of the device as disclosed herein and a method for vascularising a cell aggregate.

### TECHNICAL FIELD

The present disclosure relates broadly to a device and a method for vascularising a cell aggregate.

### BACKGROUND

Various systems for culturing cell aggregates and/or organoids (including microfluidic systems) have been proposed thus far. However, it has been recognised that these systems lack the possibility to control the aggregate position and form a controlled and consistent vasculature surrounding the whole cell aggregate.

In view of the above, there is a need to address or at least ameliorate the above-mentioned problems. In particular, there is a need to provide a device and a method for vascularising a cell aggregate that address or at least ameliorate the above-mentioned problems.

### SUMMARY

In one aspect, there is provided a device for vascularising a cell aggregate, the device comprising: a matrix region configured to contain a gel-like matrix and the matrix region having at least one opening for positioning the cell aggregate therein based on a desired three-dimensional spatial location; and one or more fluidic regions configured to contain a supporting fluid that is capable of supporting vascularisation of the cell aggregate, the one or more fluidic regions being in fluid communication with the matrix region, wherein a flow passage from the one or more fluidic regions to a gel-like matrix disposed in the matrix region is configured to allow three-dimensional vascularisation around the cell aggregate and perfusion of the vasculature once formed.

In one embodiment, the at least one or more fluidic regions comprises at least two fluidic regions.

In one embodiment, one fluidic region is disposed lateral to the matrix region on one side and the other fluidic region is disposed lateral to the matrix region on the opposite side.

In one embodiment, the flow passage is substantially free from intervening structural obstacles disposed between the one or more fluidic regions and the matrix region.

In one embodiment, the at least one opening is positioned substantially central to the matrix region.

In one embodiment, each fluidic region comprises at least two openings for facilitating introduction of the supporting fluid in each of the fluidic region.

In one embodiment, the matrix region is substantially symmetrical in shape along its longitudinal length.

In one embodiment, the at least two fluidic regions are symmetrically disposed about the matrix region.

In one embodiment, the device further comprises a gel-like matrix disposed within the matrix region.

In one embodiment, at least part of the walls defining the matrix region and the one or more fluidic regions comprise an elastomer.

In one embodiment, the device comprises an elastomer disposed on a substrate, wherein the elastomer comprises patterns formed on an open surface of the elastomer, the patterns corresponding to a layout of the matrix region and the one or more fluidic regions, and wherein the substrate substantially fluidically seals the patterns at the open surface of the elastomer to form the matrix region and the one or more fluidic regions of the device.

In one embodiment, the at least two fluidic regions are separated from one another by the matrix region.

In one aspect, there is provided a chip comprising a plurality of the device disclosed herein.

In one aspect, there is provided a method for vascularising a cell aggregate, the method comprising: proving a device comprising a matrix region configured to contain a gel-like matrix and the matrix region having at least one opening for positioning the cell aggregate therein based on a desired three-dimensional spatial location; and one or more fluidic regions configured to contain a supporting fluid that is capable of supporting vascularisation of the cell aggregate, the one or more fluidic regions being in fluid communication with the matrix region, wherein a flow passage from the one or more fluidic regions to a gel-like matrix disposed in the matrix region is configured to allow three-dimensional vascularisation around the cell aggregate and perfusion of the vasculature once formed; introducing the gel-like matrix into the matrix region; positioning a cell aggregate into the gel-like matrix via the at least one opening; and introducing the support fluid into the one or more fluidic regions.

In one embodiment, the gel-like matrix is substantially maintained in the matrix region by surface tension.

In one embodiment, the cell aggregate is positioned in the gel-like matrix such that the gel-like matrix fully surrounds the cell aggregate.

In one embodiment, the method further comprises introducing cells capable of supporting vascularisation of the cell aggregate into the one or more fluidic regions.

In one embodiment, the method further comprises introducing one or more cell types into the gel-like matrix capable of supporting vascularisation of the cell aggregate and interacting with the cell aggregate.

In one embodiment, the method further comprises vascularising the cell aggregate to obtain a three-dimensional vascularisation around the cell aggregate.

In one embodiment, the method further comprises introducing one or more test agents into the one or more fluid regions and/or the matrix region; and analysing the effect of the one or more test agents on the cell aggregate.

### DEFINITIONS

The term “cell aggregate” as used herein is to be interpreted broadly to refer to any cluster of cells that are grouped together, for example, in a three-dimensional (3D) manner, and may include (but not limited to) an aggregate of cells which may be of the same cell type such as a tumor spheroid or different cell types, such as an organoid, a biopsy etc.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals.

Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure. Unless otherwise stated or required, one or more steps may also be omitted or removed in certain embodiments and these embodiments are understood to be still within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Exemplary, non-limiting embodiments of a device and a method for vascularising a cell aggregate are disclosed hereinafter.

There is provided a device (e.g., a microfluidic device) comprising a matrix region (e.g., a main region or central channel) configured to contain a gel-like matrix; and one or more fluidic regions (e.g., supporting regions or lateral channels) in fluid communication with the matrix region, the one or more fluidic regions configured to contain a supporting fluid. In various embodiments, cells can be seeded in the one or more fluidic regions and the matrix region, and each of these regions may be configured to be seeding channels. In various embodiments, the difference between the matrix region and the fluidic region is that the matrix region has a gel-like matrix with or without cells and the fluidic region(s) have a fluid with or without cells. Advantageously, various embodiments of the device disclosed herein, in addition to having the benefits of microfluidic technology, enable the possibility to: better control the vascularisation of a cell aggregate that is seeded into the device; obtain consistency in the results among experimental replicates owing to a substantially consistent positioning of the cell aggregate inside the device; speed up imaging (for example, by either standard or high-content screening confocal microscopy); more easily extract a portion of the gel-like matrix (e.g., a 3D hydrogel) containing the cell aggregate for post-processing analysis (e.g., histology); and develop an automated process to scale up the system for high-throughput owing to the substantially consistent positioning of the cell aggregate inside the device.

In various embodiments, the matrix region comprises at least one opening configured to allow seeding/introduction of a cell aggregate into the matrix region. In various embodiments, the at least one opening is positioned substantially central to the matrix region, for example substantially in the middle of the matrix region along its longitudinal length and/or substantially in the middle of the matrix region along its width and/or substantially in the middle of the matrix region along its depth and/or a xy-plane of the matrix region and/or xz-plane of the matrix region and/or yz plane of the matrix region. In some embodiments, the opening disclosed herein may be understood as to be a three dimensional feature (e.g. a volume of space or gap) and in some other embodiments, the opening disclosed herein may be understood to be a two dimensional feature (e.g. a surface or planar feature/access). Advantageously, in various embodiments, the at least one opening allows for fixing and/or positioning of the cell aggregate in a seeding position (or a controlled location) within the matrix region. For example, the at least one opening allows positioning of the cell aggregate in the matrix region based on a desired three-dimensional spatial location.

In various embodiments, the at least one opening is accessible from the top of the device. Advantageously, this allows the cell aggregate to be seeded/introduced/inserted directly into the channels (e.g., microchannels) of the device without the need for the cell aggregate to be exposed to physico-mechanical changes that could potentially occur when cells flow through a fluidic channel.

In some embodiments, there may be more than one opening present. These other openings may lead to different portions of the matrix region and may advantageously facilitate access to these different portions of the matrix region for the positioning of cell aggregates in these regions. Accordingly, if desired, multiple cell aggregates may be positioned in different portions of the matrix region. This may advantageously maximise use of the matrix region for studies.

In various embodiments, the matrix region contains a gel-like matrix (for e.g., a gel-like matrix is disposed within the matrix region). The gel-like matrix may comprise a hydrogel, for example, an extracellular matrix (ECM)-like hydrogel, collagen gel, fibrin gel, matrigel® or other custom-made hydrogels, such as a mix of collagen gel and fibrin gel. In various embodiments, when the cell aggregate is seeded via the at least one opening, the seeding position of the cell aggregate is controlled inside the gel-like matrix. In other words, the seeding position of the cell aggregate may be controlled in position in the xy-plane, xz-plane and/or yz-plane (for e.g. controlled to be substantially central or off-centre in one or more of the planes) of the gel-like matrix. For example, the cell aggregate may be seeded in a position that is substantially central ‘in the bottom matrix region’ which facilitates vascularization/imaging. In various embodiments, the cell aggregate is fully embedded in the gel-like matrix (i.e., the cell aggregate is fully surrounded by the gel-like matrix). Advantageously, in various embodiments, the embedded positioning of the cell aggregate within the gel-like matrix allows vasculature to fully surround the cell aggregate because vasculature can reach all exterior surfaces of the cell aggregate. In various embodiments, the seeding of the cell aggregate may be controlled to the desired xyz position in a volume of gel-like matrix (e.g. hydrogel) by “hand operation” of by “robotic” operations.

In various embodiments, the at least one opening is (or plurality of openings are each) substantially symmetrical in shape. For example, the opening may be substantially ellipsoid or substantially circular in shape. Other shapes including parallelogramic shapes such as a square, rectangular, rhombus etc. can also be used. In other embodiments, the opening may be irregular in shape. It will be appreciated that any types/shapes/configurations of opening may be used as long as it is able to facilitate the introduction of a gel-like matrix into the matrix region and the seeding of the cell aggregate within the gel-like matrix. In some embodiments, the opening may have a tapered profile (e.g., tapered side profile) in the form of an opening channel that tapers from the top towards the matrix region.

In various embodiments, the one or more fluidic regions at least partially surrounds the matrix region. In various embodiments, when there is (only) one fluidic region, the fluidic region may be configured to surround (or encircle) the matrix region, either partially or fully. In various embodiments, when there is a plurality of fluidic regions present, each of the fluidic regions may at least partially surround the matrix region. In various embodiments, the length of at least one fluidic region (or each of the fluidic region) substantially spans the length of the matrix region. Advantageously, this allows the majority of the length of the matrix region to be fed by the at least one fluidic region (e.g., substantially the entire length of the gel-like matrix in the matrix region may be perfused with the supporting fluid from the at least one or more fluidic regions).

As an example, in various embodiments, there may be at least two fluidic regions wherein one fluidic region is disposed lateral to the matrix region on one side and the other fluidic region is disposed lateral to the matrix region on the opposite side (i.e., the fluidic regions flank the sides of the matrix region). In various embodiments, the at least two fluidic regions may further be symmetrically disposed about the matrix region, and the fluidic regions may be separated from one another by the matrix region. In some embodiments, there may be an even number of fluidic regions (i.e., multiples of two). Accordingly, an equal number of fluidic regions may be disposed on the two opposite lateral sides of the matrix region (i.e., separated by the matrix region) and/or the fluidic regions may be symmetrically disposed about the matrix region. Advantageously, the symmetric disposition of the fluidic regions about the matrix region may better allow the matrix region to be more evenly perfused with supporting fluid from different sides. This may in turn lead to a more even or complete three-dimensional vascularisation around the cell aggregate. In various embodiments, there may be passive liquid movement through the vasculature by pressure gradient or active liquid movement (perfusion) through the vasculature by using pumps for example (i.e., the device can be compatible with an “active” pumping system).

In various embodiments, the one or more fluidic regions contain a supporting fluid (or a fluidic medium or a perfusion medium). In various embodiments, the supporting fluid is capable of supporting vascularisation of the cell aggregate. The supporting fluid may comprise cell culture/growth medium, growth factors (e.g., including angiogenesis stimulating factors etc.), cells (e.g., endothelial cells), cytokines, nanoparticles for drug delivery, antibodies, active agents etc. In various embodiments, the supporting fluid can be directed through the gel-like matrix in the matrix region by pressure gradient and by diffusion for example. The supporting fluid can also be driven through the gel-like matrix in the matrix region by an external source (e.g., by a pump) to create a dynamic fluid flow.

In various embodiments, the matrix region is configured to substantially maintain the gel-like matrix in the matrix region. For example, when the gel-like matrix is injected into the matrix region (e.g., a dedicated region) and the gel-like matrix will substantially stay/remain in the matrix region (i.e., not flow into the fluidic regions) e.g., due to surface tension. Advantageously, in various embodiments, filling of the gel-like matrix in the fluidic regions can be avoided. On the other hand, in various embodiments, when a supporting fluid (e.g., culture media) is added into the fluidic regions, the supporting fluid can flow through the gel-like matrix, which is porous. The gel-like matrix can also be perfused by pressure gradient. In other words, the supporting fluid can enter the matrix region. In various embodiments, the supporting fluid from one fluidic region is substantially incapable of flowing to another fluidic region without passing through the matrix region when for example the fluidic regions are not configured to surround (or encircle) the matrix region.

In various embodiments, the device is configured to culture and vascularise a cell aggregate comprising various cell types. For example, the cell aggregate may at least comprise tumour cells including, but not limited to, cancer cells. As another example, a cell aggregate in the form of an organoid may be made of (or may comprise) induced pluripotent stem cells that differentiate into cells of various organs. As another further example, the device may also culture and vascularise a non-tumour organoid. In various embodiments, the cell aggregate, as well as the gel-like matrix, may include/comprise tumour cells, cancer cells, endothelial cells, stromal cells (e.g., fibroblast), organ-specific cells, immune cells (e.g., T cell, NK cell, monocytes, macrophages, Dendritic cells or the like), and/or one or more cell types capable of interacting with the cell aggregate and/or cell types capable of supporting vascularisation (including vasculature formation) of the cell aggregate. In various embodiments, the supporting fluid and/or the one or more fluidic regions may at least comprise endothelial cells (and/or other cell types capable of supporting vascularisation of the cell aggregates), immune cells, and/or cancer cells, or may comprise only cell culture medium without cells.

In various embodiments, the matrix region and the one or more fluidic regions are configured to at least allow sprouting/growth/proliferation of endothelial cells from the one or more fluidic regions to the matrix region. This may occur when the supporting fluid introduced/flowed into the one or more fluidic regions at least comprises endothelial cells. Advantageously, in various embodiments, the device is thus configured to allow vasculature formation by angiogenesis, wherein the endothelial cells seeded in the one or more fluidic regions migrate into the gel-like matrix of the matrix region and reach the cell aggregate positioned in the gel-like matrix to vascularise the cell aggregate.

In various embodiments, the device is also configured to advantageously allow vasculature formation by vasculogenesis. This may occur when endothelial cells and other supporting cells (e.g., stromal cells such as fibroblasts) are seeded in the gel-like matrix directly where the cells self-organise in a perfusable vasculature network.

In various embodiments, a flow passage from the one or more fluidic regions to the matrix region is substantially free from intervening structural obstacles such as pillars disposed between the one or more fluidic regions and the matrix region. That is, the various embodiments of the device disclosed herein is substantially free from pillars or structures that obstruct/impede/slow the flow of the supporting fluid from the one or more fluidic regions to the matrix region (e.g., the device is substantially devoid of such pillars and structures that surround the matrix region), and that disrupt the three-dimensional vascularisation around the cell aggregate. In various embodiments, the flow passage from the one or more fluidic regions to the gel-like matrix in the matrix region is thus configured to allow three-dimensional vascularisation around the cell aggregate and perfusion of the vasculature once formed. Such a flow passage may be significant in perfusing the formed vasculature with the supporting fluid (e.g., a medium containing nutrients growth factors and cells/drugs/nanoparticles to reach the cell aggregate). Advantageously, such a ‘postless’ system may add to the physiological relevance of the model (by better mimicking the physiological flow environment for vascularisation). On the contrary, having intervening pillars or posts in the flow passage (e.g., between a fluidic region and the matrix region) may not allow three-dimensional vascularisation around the cell aggregated to properly occur, undermining the physiological relevance of the model.

In various embodiments, the cross-sectional area of the flow passage (e.g., the flow passage being between the one or more fluidic regions and the matrix region) substantially spans the longitudinal cross-sectional area of the matrix region. Advantageously, this allows the majority/most of the cross-section of the matrix region to be fed by the at least one fluidic region (e.g., for the gel-like matrix of the matrix region to be perfused with the supporting fluid from the at least one or more fluidic regions). In various embodiments, the at least one fluidic region and the matrix region are disposed at approximately/substantially the same depth in the device such that the longitudinal cross-section of the at least one fluidic region (or plurality of fluidic regions) is substantially aligned with the longitudinal cross-section of the matrix region. In various embodiments, the at least one fluidic region and the matrix region are disposed at the bottom of the device.

In various embodiments, the matrix region and/or at least one of the one or more fluidic regions is substantially symmetrical in shape. Further, each of the one or more fluidic regions can be substantially symmetrical in shape. In various embodiments, the symmetry can be changed (e.g., to be no longer symmetrical) to usefully change flow across the gel-like matrix (e.g., hydrogel).

In various embodiments, line of symmetry of the device and a line of symmetry of the matrix region are substantially coincident.

In various embodiments, the cell aggregate may sink or enter the matrix region (i.e., be seeded) by microinjection and/or pipetting and/or gravity. In various embodiments, gravity can be used if the gel-like matrix (e.g., hydrogel) is low in viscosity. Otherwise (i.e., the gel-like matrix does not have low viscosity), the cell aggregate may be embedded in the gel-like matrix (e.g., hydrogel) with a standard laboratory pipette for example.

In various embodiments, the at least one opening optionally positioned substantially in the middle of the matrix region along its longitudinal length (e.g., the at least one central opening) can be configured to allow insertion of the gel-like matrix. In various embodiments, the matrix region can further comprise additional inlet/outlet openings (e.g., separate from the at least one central opening) which are configured to allow insertion and/or removal of the gel-like matrix. These additional inlet/outlet openings may usefully allow the gel-like matrix to be inserted from the additional inlet/outlet openings of the matrix region as well as from the at least one central opening in the matrix region.

In various embodiments, each of the fluidic regions may comprise at least one or a plurality of openings (or access holes) for facilitating the introduction of the supporting fluid in each of the fluidic region. In various embodiments, each fluidic region comprises at least two openings. The openings may be provided as annexures the main portion of the fluidic regions. Therefore, in various embodiments, the annexures are in fluidic communication with the main portion of the fluidic regions. For example, these openings may be attached to the main portion of the fluidic regions (e.g., the portion that is immediately adjacent to the matrix region) in the form of wells. These wells may have a tapering/tapered shape/profile (e.g., tapered side profile) that tapers from a larger/broader opening at the top to a smaller/narrower bottom.

In various embodiments, the matrix region is configured to allow culturing and growth of organoids of up to about few millimetres in size. In various embodiments, the organoids may have a size of up to about 2 mm or more. It will be appreciated that organoids of other sizes may also be obtained. For example, if larger organoids are required, a device with bigger channels (in relation to the matrix region and the fluidic regions for example) and openings to host bigger aggregates may be provided/fabricated.

In various embodiments, the device can be fabricated with an elastomer (e.g., polydimethylsiloxane (PDMS)) through a soft-lithography process and specifically by replica moulding. In devices fabricated through such methods, at least part of the walls (or surfaces) defining the matrix region and the at least one fluidic region (or a plurality of fluidic regions) may thus comprise an elastomer. Advantageously, the elastic nature of the elastomer adds to the physiological relevance of the device as it better represents the milieu or surrounding soft tissues in a biological setting in vivo during cell growth and/or vascularisation. The use of PDMS as the elastomer may also advantageously allow for the device to be fabricated with ease (e.g., in a relatively short span of time and in a relatively straightforward manner) and also allow for the device to be imaged due to the transparency of PDMS.

The elastomer used to fabricate the device (e.g., the top part of the device) may be natural rubbers, styrene-butadiene block copolymers, polyisoprene, polybutadiene, ethylene propylene rubber, ethylene propylene diene rubber, silicone elastomers, fluoroelastomers, polyurethane elastomers, nitrile rubbers or the like or combinations thereof. In various embodiments, the elastomer contains a silicon containing elastomer and thus contain silicon element. In various embodiments, the fabrication procedure may be modified, for example, by changing the material (for example, from PDMS to a polymeric (plastic) material, such as thermoplastic materials, including but not limited to cyclic olefin copolymer (COC), polypropylene (PP), and polymethylmethacrylate (PMMA)) and changing the fabrication procedure accordingly.

In various embodiments, the device comprises an elastomer disposed on/coupled to a substrate, wherein the elastomer comprises patterns formed on an open surface of the elastomer, the patterns corresponding to a layout of the matrix region and the one or more fluidic regions, and wherein the substrate fluidically seals (e.g., substantially hermetically sealing) the patterns at the open surface of the elastomer to form the matrix region and the one or more fluidic regions of the device. In various embodiments, the coupling of the elastomer to the substrate forms a closed flow passage from the at least one fluidic region to the matrix region. The coupling of the elastomer to the substrate may be carried out by increasing the adhesiveness of the elastomer and the substrate to each other, and this may be carried out by e.g., activating the contacting surfaces of the elastomer and the substrate through e.g., plasma activation. It may also be possible to apply adhesives on the contacting surfaces of the elastomer and the substrate before contacting them together.

The substrate may be elastic, non-elastic, rigid or non-rigid. For example, the substrate may also comprise an elastomer (e.g., a silicon containing elastomer such as PDMS or the like). In various embodiments, the substrate is substantially rigid and non-elastic and comprises an inorganic oxide (e.g., silicon dioxide). In various embodiments, the substrate is a glass substrate. Accordingly, in various embodiments, the side walls of the channels of the flow passage are formed by the elastomer and the bed of the channels is formed by the glass substrate. In various embodiments, the elastomer and the substrate both contain at least one common element. For example, the elastomer and the substrate both contains silicon as an element.

In various embodiments, the device is configured to be suitable for imaging with a standard or high-content screening confocal microscopy for example.

There is also provided a chip comprising a plurality of the device provided herein. The chip may comprise a plurality of devices arranged in an A×B array, where A and B are integers that are independently selected from 1 to 10. As examples, the chip may comprise a 3 by 1 array or a 5 by 1 array. Advantageously, in various embodiments, the chip comprising the plurality of the device can increase throughput, i.e., to culture and/or vascularise multiple cell aggregates simultaneously.

There is also provided a method of culturing and/or vascularising a cell aggregate, the method comprising: providing/introducing/positioning a gel-like matrix in a matrix region (e.g., a main region or central channel) of the device provided herein; and providing/introducing/positioning a supporting fluid in one or more fluidic regions (e.g., supporting regions or lateral channels) in the device provided herein.

In various embodiments, the method further comprises seeding/introducing/positioning a cell aggregate into the matrix region via at least one opening of the matrix region. In various embodiments, the at least one opening is substantially in the middle of the matrix region along its longitudinal length. Advantageously, in various embodiments, the method allows fixing and/or positioning the cell aggregate in a seeding position (or a controlled location) within the matrix region via the at least one opening. That is, the at least one opening advantageously allows positioning of the cell aggregate in the matrix region based on a desired three-dimensional spatial location.

In various embodiments, the method comprises positioning the cell aggregate substantially in the portion of the matrix region that is in the path of the flow passage or fed by the at least one fluidic region. Advantageously, this allows the cell aggregate to be properly and adequately supplied with the supporting fluid and/or three-dimensional vascularisation to be formed around the cell aggregate. In various embodiments, the cell aggregate is positioned in the matrix region such that it is in substantial alignment with the cross-section of the flow passage or the at least one fluidic region (e.g., substantially in the middle of the cross-section of the flow passage or the at least one fluidic region or the matrix region). In various embodiments, the at least one fluidic region and the matrix region are disposed at the bottom of the device and the cell aggregate is positioned nearer to the bottom of the device.

In various embodiments, the method comprises seeding/introducing/inserting the cell aggregate directly into the channels (i.e., microchannels) from a top surface of the device provided herein. Advantageously, the cell aggregate would not be exposed to physico-mechanical changes that could potentially occur when cells flow through a fluidic channel.

In various embodiments, the method further comprises introducing/inserting the gel-like matrix into the matrix region. The gel-like matrix may be a hydrogel, for example, an extracellular matrix (ECM)-like hydrogel, collagen gel, fibrin gel, matrigel® or other custom-made hydrogels, such as a mix of collagen gel and fibrin gel. In various embodiments, when the cell aggregate is seeded/introduced/inserted via the at least one opening, and when the cell aggregate is disposed in the gel-like matrix, the seeding position of the cell aggregate is controlled inside the gel-like matrix. In other words, the seeding position of the cell aggregate may be controlled in position in the xy-plane, xz-plane and/or yz-plane of the gel-like matrix (for e.g. controlled to be substantially central or off-centre in one or more of the planes). In various embodiments, the cell aggregate is fully embedded in the gel-like matrix (i.e., the cell aggregate is fully surrounded by the gel-like matrix). For example, the cell aggregate is disposed (or positioned) in the gel-like matrix such that the gel-like matrix fully surrounds the cell aggregate (e.g., the gel-like matrix substantially buries the cell aggregate). Advantageously, in various embodiments, the positioning of the cell aggregate within the gel-like matrix allows vasculature to fully surround the cell aggregate because vasculature can reach all exterior surfaces of the cell aggregate. If more than one opening leading to the matrix region is present, additional cell aggregates may also be inserted/introduced into different parts of the matrix region via these other openings if desired.

In various embodiments, the method further comprises providing/introducing a supporting fluid (or a fluidic medium or a perfusion medium). In various embodiments, the supporting fluid is capable of supporting vascularisation of the cell aggregate. The supporting fluid may comprise a cell culture/growth medium, growth factors (e.g., including angiogenesis stimulating factors etc.), cells (e.g., endothelial cells), cytokines, nanoparticles for drug delivery and antibodies etc. In various embodiments, the method further comprises directing the supporting fluid through the gel-like matrix in the matrix region by diffusion and/or convection for example. The supporting fluid can also be driven through the gel-like matrix in the matrix region by an external source (e.g., by a pump) to create a dynamic fluid flow.

In various embodiments, the method comprises substantially maintaining a gel-like matrix in the matrix region. In various embodiments, the method comprises injecting the gel-like matrix in the matrix region (i.e., a dedicated region) and substantially maintaining the gel-like matrix in the matrix region (i.e., not allowing the gel-like matrix to flow into the fluidic regions) e.g., by surface tension. Advantageously, in various embodiments, filling of the gel-like matrix in the fluidic regions can be avoided. In various embodiments, the method further comprises adding a supporting fluid into fluidic regions and flowing the supporting fluid through the gel-like matrix, which is porous. The method may also comprise perfusing the gel-like matrix by a pressure gradient. In other words, the supporting fluid may enter the matrix region. In various embodiments, the method further comprises flowing the supporting fluid from one fluidic region to another fluidic region by passing through the matrix region.

In various embodiments, the method may comprise introducing/incorporating various cell types into the device described. The method may further comprise culturing and vascularising such cell type(s). As one example, the method may comprise introducing/incorporating cells for example, but not limited to, tumour/cancer cells in the cell aggregate. As another example, the method may comprise incorporating a cell aggregate in the form of an organoid that is made of (or comprise) induced pluripotent stem cells that differentiate into cells of various organs. As another further example, the method may comprise introducing/incorporating a non-tumour organoid. In various embodiments, the method may comprise incorporating/introducing but not limited to tumour cells, endothelial cells, stromal cells (e.g., fibroblast), organ-specific cells, and immune cells (e.g., T cell, NK cell, monocytes, macrophages, Dendritic cells) (i.e., one or more cell types capable of supporting vascularisation (including vasculature formation) and/or interacting with the cell aggregate) into the gel-like matrix. In various embodiments, the method may additionally comprise incorporating/introducing but not limited to endothelial cells (and/or other cells capable of supporting vascularisation of the cell aggregate), immune cells, and/or cancer cells in the supporting fluid and/or the one or more fluidic regions, or may not comprise incorporating/introducing cells in the supporting fluid and/or the one or more fluidic regions (e.g., the supporting fluid and/or the one or more fluidic regions may only comprise cell culture medium without cells).

In various embodiments, the method comprises allowing the endothelial cells to sprout/grow/proliferate from the one or more fluidic regions to the matrix region. In various embodiments, this may be possible when the supporting fluid introduced/flowed into the one or more fluidic regions at least comprises endothelial cells. In various embodiments, the method comprises seeding the endothelial cells in the one or more fluidic regions, allowing the endothelial cells to migrate into the gel-like matrix of the matrix region and reach the cell aggregate positioned in the gel-like matrix to vascularise the cell aggregate. Thereby, in various embodiments, the method advantageously allows vasculature formation by angiogenesis.

In various embodiments, the method comprises seeding endothelial cells and other supporting cells (e.g., stromal cells such as fibroblasts) in the gel-like matrix directly. In various embodiments, the method allows the cells to self-organise in a perfusable vasculature network. Thereby, in various embodiments, the method advantageously allows vasculature formation by vasculogenesis.

In various embodiments, the method further comprises vascularising the cell aggregate to obtain a three-dimensional vascularisation around the cell aggregate.

In various embodiments, the method comprises allowing the cell aggregate to sink or enter the matrix region (i.e., be seeded) by microinjection and/or pipetting and/or gravity. In various embodiments, the cell aggregate may enter the matrix region by gravity if the gel-like matrix (e.g., hydrogel) is low in viscosity. Otherwise (i.e., the gel-like matrix does not have low viscosity), the cell aggregate may be embedded in the gel-like matrix (e.g., hydrogel) with a standard laboratory pipette for example.

In various embodiments, the method may comprise introducing/inserting the gel-like matrix into the matrix region via the at least one opening optionally positioned substantially in the middle of the matrix region along its longitudinal length (i.e., the at least one central opening). The method may also comprise introducing/inserting the gel-like matrix into the matrix region via additional inlet/outlet openings (separate from the at least one central opening) which are configured to allow insertion and/or removal of the gel-like matrix. In various embodiments, the additional inlet/outlet openings can usefully allow the gel-like matrix to be inserted from the additional inlet/outlet openings of the matrix region as well as from the at least one central opening in the matrix region.

In various embodiments, the method allows culturing and growing of organoids of up to about few mm. In various embodiments, the method comprises growing organoids with a size of up to about 2 mm or more. It will be appreciated that in various embodiments, the method allows the gel-like matrix (e.g., hydrogel) on the top and bottom regions of the organoid/aggregate to form a vasculature around the organoid/aggregate.

In various embodiments, the method further comprises introducing one or more test agents into the one or more fluidic regions and/or the matrix region and analysing the effect of the one or more test agents on the cell aggregate. In various embodiments, test agents may broadly refer to therapeutic agents such as drug compounds (e.g., to perform drug compound screening), for example Sorafenib, or cells (e.g., to test cell therapy), for example TCR-engineered T cells, CD133 CAR-T cells or the like, antibodies (e.g., to test antibody therapy), for example anti-PD-1 or anti-PD-L1 or the like, mRNAs (e.g. mRNA vaccines), or antisense oligonucleotides.

In various embodiments, the method further comprises imaging the cell aggregate at different time intervals e.g., to observe vascularisation and/or the effect of the test agents on the cell aggregate (including the degree/progress of vascularisation in some cases).

An exemplary device is described below for illustrative purposes. In one example, there is provided the following: a device (e.g., a microfluidic device) for culturing and vascularising cell aggregates comprising of 3 compartments; wherein two compartments comprise of liquid and, one compartment comprises of extracellular matrix (ECM)-like hydrogel and a circular opening in the middle of the gel region to precisely control the position of a cell aggregate inside the matrix.

In the above example, the microfluidic device is devoid of using pillars to separate the fluidic region (e.g., the two compartments comprising liquid) from the hydrogel region (e.g., the compartment comprising ECM-like hydrogel). Further, the device allows the culture of cell aggregates (e.g., in the form of organoids) with the possibility to precisely control the position (the spatial location) of the cell aggregates in the 3D matrix and with vascularisation (i.e., with the presence of a surrounding microvasculature). In the above example, the device allows substantially controlled vasculature formation and has the capability to create a vasculature network around the cell aggregate because the ECM-like hydrogel substantially fully embeds the cell aggregates and/or the vasculature can reach the cell aggregate due to the device layout.

In the above example, the cell aggregates develop an internal vasculature and are fully surrounded by a 3D extracellular matrix.

In the above example, the device is capable of vascularising a cell aggregate with endothelial cells seeded in lateral channels. In the above example, the vasculature can not only reach the bottom of the spheroid but around the spheroid due to the device layout. In the above example, the endothelial cells can surround the whole spheroid to perform vascularisation.

In the above example, the ECM-like hydrogel may comprise (but not limited to) endothelial cells and stromal cells. The presence of endothelial cells and stromal cells in the matrix as well as the device layout can advantageously allow the growth of a vasculature completely surrounding the cell aggregate.

In the above example, injecting the hydrogel without the need of pillars and growing the vasculature network without letting endothelial cells migrate from parallel channels (e.g., relying on endothelial cells and stromal cells in the matrix for the vascularisation instead) can result in a more efficient and faster vasculature formation compared to having the endothelial cells in the lateral channels.

In the above example, the overall dimensions of the device may allow the culture and growth of organoids up to about ˜2 mm.

In the above example, the device can be fabricated with an elastomer (e.g., PDMS) through a soft-lithography process and specifically by replica moulding. The elastomer may therefore contain patterns that correspond to the layout of the matrix region and the one or more fluidic regions. The fabrication procedure of the device can be modified, for example, by changing the material (for example, from PDMS to a plastic material, such as thermoplastic materials) and changing the fabrication procedure accordingly. The patterned elastomer may then be attached/disposed on a substrate (e.g., a non-elastic or a rigid substrate (e.g., a glass substrate) to form the entire device.

Accordingly, there is also provided a method of fabricating/making the device, the method comprising forming patterns corresponding to a layout of the matrix region and the one or more fluidic regions on an open surface of an elastomer (e.g., by etching or using a mould); coupling a substrate to the open surface of the elastomer to substantially sealing (e.g., substantially fluidically sealing or hermetically sealing) the patterns at the open surface of the elastomer thereby forming the matrix region and the one or more fluidic regions of the device. The method may further comprise activating the substrate and elastomer surfaces prior to coupling the substrate and the elastomer together via their activated surfaces together. In various embodiments, activation of the substrate and elastomer surfaces may comprise subjecting the substrate and elastomer surfaces to plasma treatment. If desired, an adhesive may also be applied on the surfaces of the substrate and elastomer to facilitate their coupling together.

In various embodiments, the device can be modified to have high throughput by making an array of such devices (e.g., to form a chip).

In various embodiments, the device may be imaged, for example, by standard or high-content screening confocal microscopy.

Various embodiments of the present disclosure can advantageously provide a device and a method to be used to study vasculature-tissue interactions.

### BRIEF DESCRIPTION OF FIGURES

FIG. 1A is a three-dimensional (3D) rendering of a chip in accordance with various embodiments disclosed herein. FIG. 1B is a schematic top view drawing of a chip, in accordance with various embodiments disclosed herein. FIG. 1C is a detail view drawing of portion X of FIG. 1B (4:1), showing a device on a chip, in accordance with various embodiments disclosed herein. FIG. 1D is a schematic drawing illustrating an isometric view of a device on a chip, in accordance with various embodiments disclosed herein. FIG. 1 E is a cross-sectional view diagram of a device taken along the line Y-Y′ of FIG. 1D, in accordance with various embodiments disclosed herein. FIG. 1F is a photograph of a chip with devices fabricated with PDMS (polydimethylsiloxane) bonded to a microscope glass slide in accordance with various embodiments disclosed herein. FIG. 1G is a zoom photo of a circular opening located substantially in the middle of a matrix region of a PDMS device, in accordance with various embodiments disclosed herein.

FIG. 2 is a schematic drawing of a mold for fabricating PDMS devices, in accordance with various embodiments disclosed herein.

FIG. 3 is a schematic drawing of another chip, in accordance with various embodiments disclosed herein.

FIGS. 4A and 4B are schematic cross-sectional diagrams illustrating a process of introducing an organoid into a device, in accordance with various embodiments disclosed herein.

FIG. 5 is a diagram showing an exemplary workflow for a 3D in vitro vascularised tumour model, in accordance with various embodiments disclosed herein.

FIG. 6A shows images of HepG2 (GFP, green) and Hep3b (GFP, green) liver tumour cell lines that were used to form monoculture (only liver cancer cells) or triculture spheroids (liver cancer cell co-cultured with stellate cells (LX2) and endothelial cells (RFP-HLSEC)), which, after 5 days, were seeded into devices in an ECM containing GFP-HUVEC and NHLF. Images of the control (“No Spheroid”) show GFP-HUVEC and NHLF that were seeded into devices. The scale bars represent 200 μm. FIG. 6B shows a graph showing vasculature coverage (%) against days post-seeding for each condition. Mean and SEM are shown. **P<0.01.

FIG. 7A is a graph showing spheroid growth over time with and without vasculature. FIG. 7B shows representative fluorescence images, as well as expanded images of the boxed portions of the respective fluorescence images, from spheroids and DRAQ7 quantification. FIG. 7C is a graph showing quantification of DRAQ7 in the spheroids. Mean and SEM from 5 samples are shown. FIG. 7D is a graph showing expression of the proliferation marker Ki-67 in vascularised spheroids relative to spheroids grown in ECM without vasculature. FIGS. 7E and 7F are graphs showing expression of proliferation marker Ki-67 in vascularised spheroids and spheroids grown in ECM without vasculature, in the core and outer regions of the spheroids respectively. FIG. 7G is a representative fluorescence image showing the distribution of the proliferation marker Ki-67 in a HepG2 monoculture spheroid with vasculature. FIG. 7H is a representative fluorescence image showing the distribution of the proliferation marker Ki-67 in a HepG2 triculture spheroid with vasculature. FIG. 7I is a graph showing the extent of apoptosis in the vascularised spheroids relative to non-vascularised spheroids, detected by immunofluorescence using Caspase-3 antibody. FIGS. 7J and 7K are graphs showing the extent of apoptosis in the vascularised spheroids relative to non-vascularised spheroids, in the core and outer regions of the spheroids respectively. FIG. 7L is a representative fluorescence image showing the extent of apoptosis in a HepG2 triculture spheroid without vasculature. FIG. 7M is a representative fluorescence image showing the extent of apoptosis in a Hep3b triculture spheroid with vasculature. FIG. 7N is a graph showing the extent of hypoxia in the vascularised spheroids relative to non-vascularised spheroids, measured using anti-Hif1a (Hypoxia-inducible factor-1 alpha) antibody. FIGS. 7P and 7Q are graphs showing the extent of hypoxia in the vascularised spheroids relative to non-vascularised spheroids, in the core and outer regions of the spheroids respectively. FIG. 7R is a representative fluorescence image showing the extent of hypoxia detected in a HepG2 triculture spheroid without vasculature. FIG. 7S is a representative fluorescence image showing the extent of hypoxia detected in a HepG2 triculture spheroid with vasculature. Fluorescence images were acquired using the same acquisition parameters in all the conditions and representative images to show the significant changes are shown. Data are represented as mean±SEM. The scale bars represent 100 μm, *P<0.05, **P<0.01, *** P<0.001, ****P<0.0001.

FIG. 8 is a graph showing vasculature coverage close to a spheroid (or adjacent to a spheroid, wherein “adjacent” means within a distance of 350 μm from the spheroid) and distant from the spheroid (wherein “distant” means more than 350 μm from the spheroid) across different conditions. Data are represented as mean±SEM. *P<0.05, **P<0.01.

FIG. 9A shows representative fluorescence images of vascularised and non-vascularised Hep3b monoculture and triculture spheroids subjected to Sorafenib (against a control), taken on Day 0 (starting of drug treatment) and Day 5 post-treatment. FIG. 9B is a graph showing the GFP signal detected for vascularised and non-vascularised Hep3b monoculture spheroids and Hep3b triculture spheroids over time subjected to Sorafenib treatment, based on 3 independent replicates. The scale bars represent 200 μm.

FIG. 10A is a graph showing DRAQ7 signals in HepG2 monoculture and triculture spheroids seeded into devices with and without vasculature and which were treated with TCR T cells or Mock T cells. FIG. 10B shows representative fluorescence images of HepG2 monoculture and triculture spheroids seeded into devices with and without vasculature and which were treated with TCR T cells or Mock T cells used for the DRAQ7 quantification. The scale bars represent 200 μm. FIGS. 10C and 10D are graphs showing DRAQ7 signals in Hep3b monoculture and triculture spheroids seeded into devices with and without vasculature which were treated with CD133 CAR-T cells. FIG. 10E is a graph showing CD133 CAR-T cell count in devices seeded with Hep3b monoculture and triculture spheroids with and without vasculature. FIG. 10F shows representative fluorescence images of vascularised and non-vascularised Hep3b monoculture and triculture spheroids treated with CD133 CAR-T cells or mock cells.

FIGS. 11A and 11B show images taken on day 3 of a vascularised patient biopsy (the biopsy obtained from an ovarian cancer patient) which was cultured in a device with endothelial cells (HUVEC) and fibroblasts (NHLF) in accordance with various embodiments disclosed herein.

### EXAMPLES

Example embodiments of the disclosure will be better understood and readily apparent to one of ordinary skill in the art from the following discussions and if applicable, in conjunction with the figures. It should be appreciated that other modifications related to structural and biological changes may be made without deviating from the scope of the invention. Example embodiments are not necessarily mutually exclusive as some may be combined with one or more embodiments to form new exemplary embodiments. The example embodiments should not be construed as limiting the scope of the disclosure.

The following examples describe a device (e.g., a microfluidic device) to culture and vascularise a cell aggregate (or an organoid or a patient biopsy) in a controlled location within a gel-like matrix (e.g., a 3D hydrogel matrix). A plurality of such devices can be arranged in a chip in the form of an array.

In the following examples, each microfluidic device is designed and prototyped with three different regions (or compartments), two regions (referred to as fluidic regions) for containing a supporting fluid (or liquid) and one region (referred to as a matrix region) for containing a gel-like matrix such as an extracellular matrix (ECM)-like hydrogel. The matrix region presents a circular opening (or openings with other shapes) to precisely control the position of a cell aggregate inside the matrix and to consistently form a vascular network within the gel-like matrix such as an ECM-like hydrogel.

Device

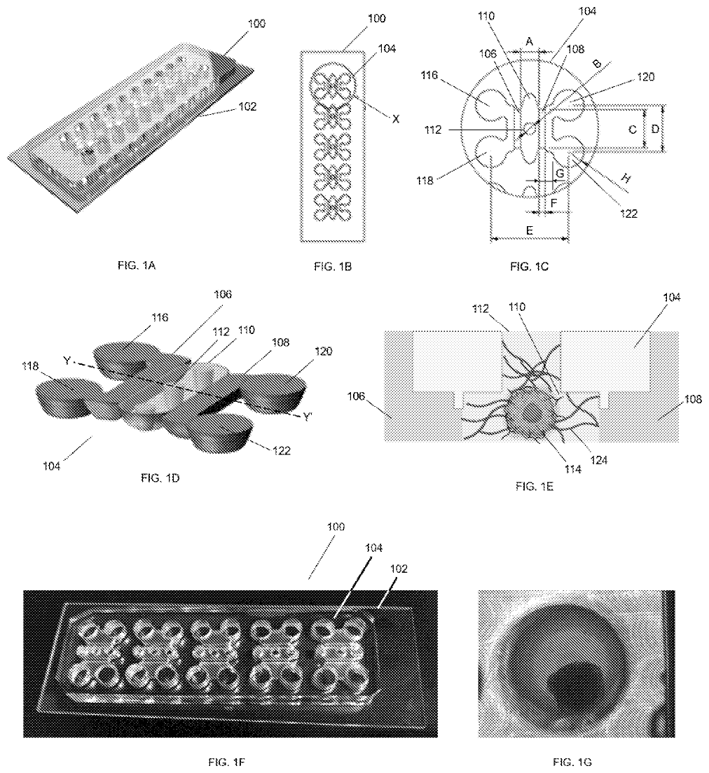

FIG. 1A shows a three-dimensional (3D) rendering of a chip 100 with a microscope glass slide 102 being its base. The chip 100 comprises a plurality of devices 104 (see FIG. 1B) which are arranged in a 5 by 1 array on the microscope glass slide 102.

FIG. 1C shows a detail view drawing of portion X of FIG. 1B, showing one of the devices 104. As shown in FIG. 1C, the device 104 (e.g., a microfluidic device) is designed with three different regions (or compartments), two regions (fluidic regions 106 and 108) for containing a supporting fluid (or liquid) and one region (matrix region 110) for containing a gel-like matrix such as an extracellular matrix (ECM)-like hydrogel. See also FIG. 1D which shows an isometric view of the device 104 and FIG. 1E which shows a cross-sectional view diagram of the device 104 taken along line Y-Y′ of FIG. 1D.

As shown in FIGS. 1C, 1D, and 1E, the matrix region 110 comprises a circular opening 112 which is accessible from the top surface of the device 104. The circular opening 112 allows precise control the position of a cell aggregate 114 inside the gel-like matrix (see FIG. 1E), e.g., by hand or by automation (such as with the use of a liquid handler). That is, the cell aggregate 114 can be seeded in a controlled position of the gel-like matrix, as shown in FIG. 1E.

The fluidic regions 106 and 108 on the other hand each comprise two openings, i.e., openings 116 and 118 for fluidic region 106 and openings 120 and 122 for fluidic region 108. The openings 116, 118, 120, 122 are provided as annexures to the main portion (i.e., the part adjacent to the matrix region that allows the supporting fluid to directly perfuse the gel-like matrix of the matrix region) of the fluidic regions 106 and 108. These annexures are in the form of wells that have a tapered profile (i.e., tapered side profile) tapering from a larger/broader opening at the top to a smaller/narrower bottom.

These openings of the fluidic regions 106 and 108 allow the supporting fluid (for example, cell culture media) to be introduced into and/or be removed from the fluidic regions 106 and 108. These openings can also act as a connection point to the external environment or external components, for example, tubes, pumps, or additional devices.

In this example, the device 104 has the following dimensions (see FIG. 1C): the width of the matrix region 110 (see A) is 2.4 mm; the diameter of the circular opening 112 (see B) is 1.5 mm; the length of the fluidic regions 106, 108 parallel to the matrix region (see C) is 5 mm; the direct distance between the centre of one opening of the fluidic region to the centre of the other opening of the same fluidic region (e.g., the distance between opening 116 to opening 118, and the distance between opening 120 to opening 122) (see D) is 6 mm; the direct horizontal distance between the centre of one opening of one fluidic region to the centre of an opening of the other fluidic region (e.g., the distance between opening 116 to opening 120, and the distance between opening 118 to opening 122) (see E) is 10 mm; the width of the regions (see F) is 0.8 mm, while the width of the complete fluidic channel (see G) is 1.8 mm; and the diameter of the openings (or inlet/outlet ports) of the whole fluidic channel (e.g., openings 116, 118, 120, 122) (see H) is 4.0 mm. In the above example, while the device 104 may have the dimensions described above, it will be appreciated that the dimensions are not limited as such and can be changed e.g., depending on the application.

The device 104 as shown in FIGS. 1C, 1D, and 1E is configured to maintain or keep the gel-like matrix, such as an ECM-like matrix, in the matrix region by surface tension. Further, as shown in FIG. 1E, the device 104 is configured such that the flow passage from the fluidic regions 106, 108 to the matrix region 110 is substantially free from intervening obstacles (e.g., pillars).

In the device 104 as shown in FIGS. 1C and 1D, the circular opening 112 is substantially in the middle of the matrix region 110 along the longitudinal length of the device 104. Positioning the circular opening 112 substantially in the middle of the matrix region 110 allows a precise control the cell aggregate seeding position in the device 104.

The device 104 can allow cells, such as endothelial cells, to enter the fluidic regions 106 and 108 (e.g., when present in the supporting fluid introduced into the fluidic regions 106 and 108) and/or the matrix region 110 (e.g., when the endothelial cells are seeded in the gel-like matrix directly). In so doing, the vasculature as well as cell aggregate layout can have different spatial distributions. Namely, vasculature can form by self-assembling of endothelial cells already inside the gel-like matrix containing the cell aggregate to completely surround the cell aggregate in all directions; and/or vasculature can form by sprouting of endothelial cells from the fluidic regions into the gel-like matrix (e.g., ECM-like matrix) to mimic angiogenesis towards the cell aggregate (e.g., to mimic newly vascularised tumours). For the latter, see vasculature 124 of FIG. 1E.

The central circular opening 112 of the device 104 further allows insertion of a cell aggregate from the top of the device 104 directly into the matrix region 110 without the need for them to be exposed to physico-mechanical changes that could potentially occur when cells are made to flow through a fluidic channel. The central circular opening 112 of the device 104 also allows the extraction of the sample (e.g., cell aggregate, an organoid, or a patient biopsy) if needed, to conduct further investigations such as histology, flow cytometry, and omics studies.

Fabricating the Device

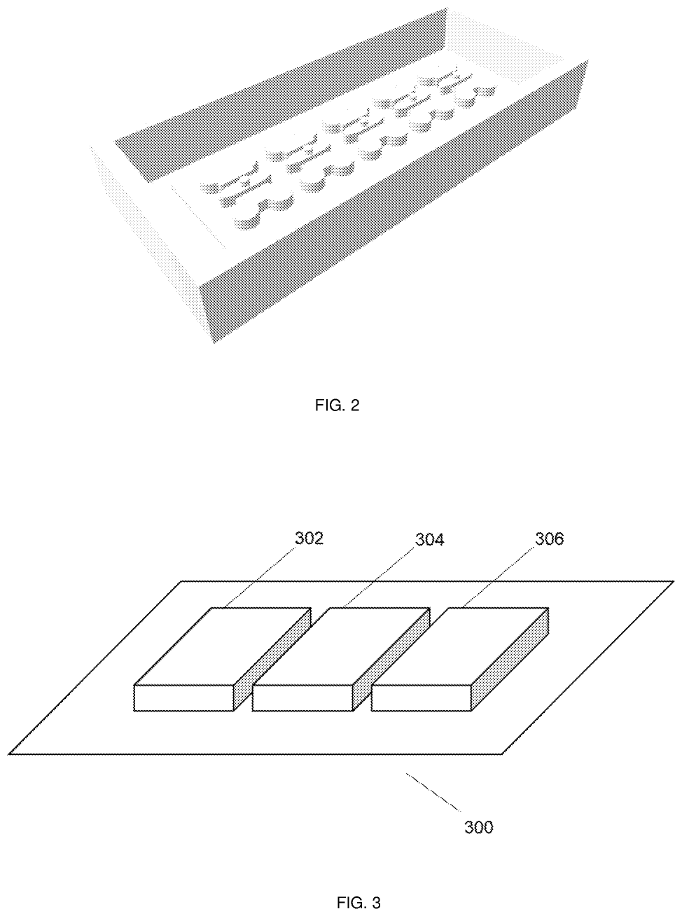

The devices 104 described with reference to FIGS. 1C and 1D for example can be fabricated with polydimethylsiloxane (PDMS) through a soft-lithography process and specifically by replica moulding. FIG. 1F shows a photograph of PDMS devices bonded to a microscope glass slide (compare microscope glass slide 102 described with reference to FIG. 1A), FIG. 1G shows a zoom photo of a circular opening located substantially in the middle of a matrix region of a PDMS device (compare central circular opening 112 described with reference to FIG. 1C), and FIG. 2 shows a schematic drawing of a mold for fabricating PDMS devices.

An exemplary protocol for device fabrication can comprise the steps of: (i) casting PDMS into a mould (e.g., by first mixing the PDMS elastomer with a PDMS curing agent in a plastic cup, then using a vacuum machine to remove all air bubbles before pouring the PDMS mix into the mould); (ii) using the vacuum machine to remove all air bubbles thoroughly; (iii) placing the mould in an 80° C. oven for about 1 hour 45 minutes; (iv) using a pair of tweezers, removing the cured PDMS from the mould; (v) placing the cured PDMS into a petri dish, and placing in the 80° C. oven overnight; (vi) cutting out the PDMS replicas and punching holes accordingly; (vii) using tape to remove any dirt or other particles from both sides of the PDMS chip; and (viii) autoclaving the PDMS replicas in a pipette box.

After the PDMS replica is prepared, the PDMS replica can be bonded to a glass coverslip. Bonding of the PDMS replica to a glass coverslip can comprise the steps of: cleaning glass coverslips with acetone, followed by isopropanol, and then 70% ethanol; using tape to clean dust off surface of PDMS and glass slide; cleaning the inside of a vacuum chamber with ethanol and making sure it is dry; performing plasma treatment on PDMS replicas and glass cover slips; bonding glass to PDMS replica; and placing the bonded chip into an oven at 70° C.

Chip Modification

In the examples described above, a chip comprises one array of devices. See FIG. 1B for example.

The chip can be modified to increase throughput for example. One example of a modified chip 300 is shown in FIG. 3 which shows the chip 300 comprising three (i.e., a plurality of) array of devices 302, 304, and 306. In the chip 300, each array 302, 304, and 306 comprises a 5 by 1 array of devices (similar to the 5 by 1 array of devices described with reference to FIG. 1B) and each device of the arrays 302, 304, and 306 is substantially similar to the device 104 described with reference to FIGS. 1C and 1D for example. The array 302, 304, and 306 are each bonded onto a microscope glass slide 308 to form the chip 300. The chip 300 with the microscope glass slide 308 as its base can be placed onto a plate holder for example for downstream analytics.

Introducing Cell Aggregates and/or Organoids into Devices

In the examples described above, a matrix region of a device comprises an opening which is substantially in the middle of the matrix region along its longitudinal length. See circular opening 112 of FIG. 1C for example.

The opening mentioned above allows cell aggregates to be introduced into a device by a number of ways. For example, the cell aggregates can be introduced into the device via the opening by one of: microinjection, pipetting and/or gravity.

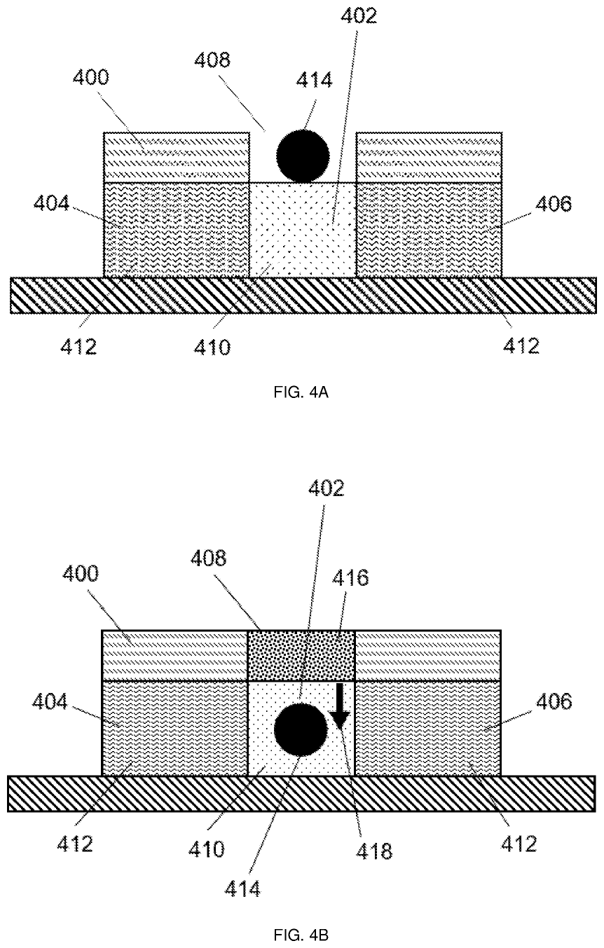

FIGS. 4A and 4B are schematic cross-sectional diagrams illustrating one example of a process of introducing an organoid into a device by pipette injection. The device 400 shown in FIGS. 4A and 4B comprises a matrix region 402 (compare matrix region 110 described with reference to FIG. 1C) and two fluidic regions 404 and 406 (compare fluidic regions 106 and 108 described with reference to FIG. 1C). The matrix region 402 provides an opening 408 (compare circular opening 112 described with reference to FIG. 1C) through which an organoid can be introduced.

In this example, a gel-like matrix 410 is present in the matrix region 402 and a supporting fluid 412 is present in the fluidic regions 404 and 406. An organoid 414 with a size of about 300 μm to about 500 μm is introduced into the device 400 via the opening 408 (see FIG. 4A), and thereafter, additional gel-like matrix 416 (e.g., collagen or other types of hydrogel such as fibrin solution and/or a mix of collagen solution, fibrin solution or the like) is introduced into the device, also through the opening 408 (see FIG. 4B). As shown in FIG. 4B, the introduction of the additional gel-like matrix 416 through the opening 408 causes the organoid 414 to sink into the gel-like matrix 410 present in the matrix region 402 (see arrow 418). In this way, the opening 408 allows the organoid 414 to be embedded in the gel-like matrix 410 with a substantially precise control of its position.

Exemplary Workflow for 3D In Vitro Vascularised Tumour Model

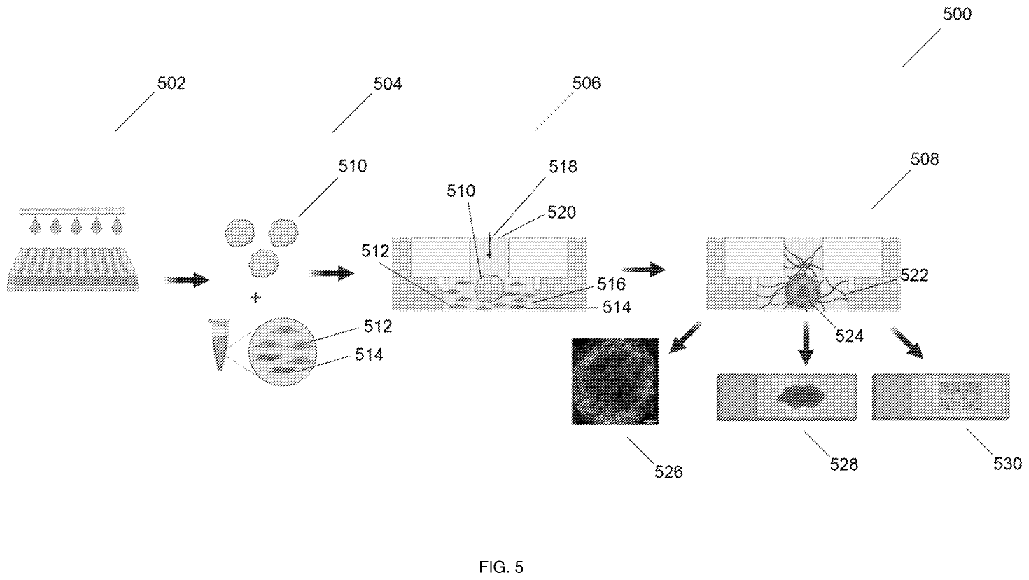

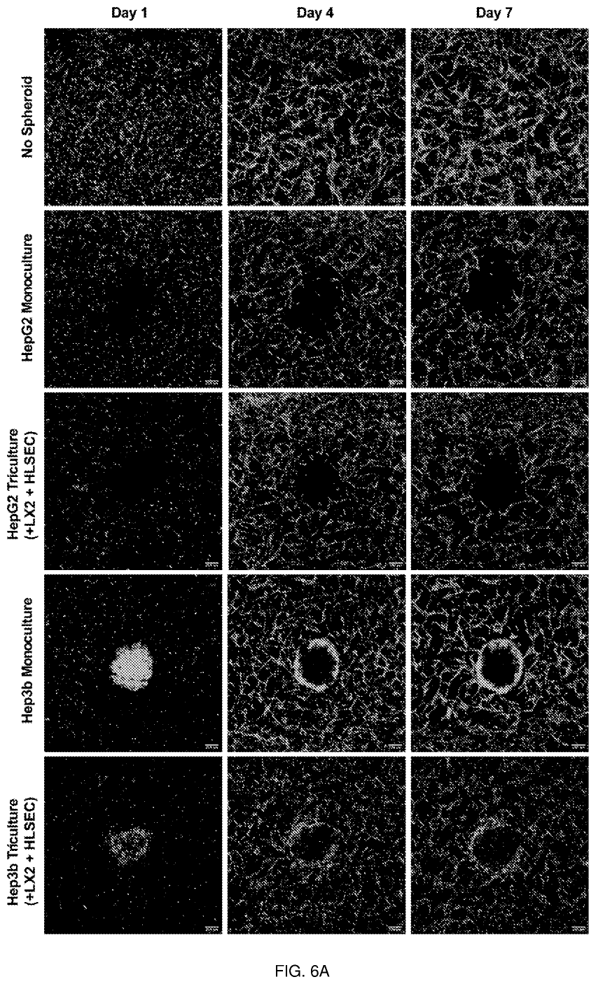

FIG. 5 is a diagram showing an exemplary workflow for a 3D in vitro vascularised tumour model. The workflow 500 shown in FIG. 5 comprises step 502, step 504, step 506, and step 508.

At step 502, tumour spheroids derived from HepG2 and Hep3b cell lines are formed using the hanging drop technique.

After 5 days, at step 504, spheroids (or cell aggregates) 510 are formed. Separately, a mixture containing endothelial cells (e.g., HUVEC (Human Umbilical Vein Endothelial Cells) and human induced pluripotent stem cell-derived endothelial cell (hiPSC-EC)) 512 and fibroblasts (e.g., NHLF (Normal Human Lung Fibroblasts)) 514 are prepared.

At step 506, the spheroids 510 are seeded into a middle channel 516 of a device (compare matrix region 110 described with reference to FIG. 1C) in an ECM (extracellular matrix) containing endothelial cells (e.g., HUVEC, hiPSC-EC) 512 and fibroblasts (e.g., NHLF) 514. See arrow 518 showing that a spheroid 510 is seeded into a middle channel via an opening 520 (compare circular opening 112 described with reference to FIG. 1C).

After 7 days, at step 508, vasculature 522 forms spontaneously around a tumour 524 (formed from spheroids 510 seeded into the device at step 506).

The prepared 3D in vitro vascularised tumour model can subsequently be used for various applications. Such applications can include analysing the effect of one or more test agents (e.g., through drug treatment or cell therapy) on the tumour 524 formed. Other applications can include analysing the tumour 524 formed via immunofluorescence (live/fixed) 526, histology characterisation 528 (e.g., to analyse cellular spatial organisation in the tumour 524), scRNA sequencing and digital spatial proteomics/transcriptomics 530.

Vasculature Formation over Time

Based on the above exemplary workflow, vasculature formation in the device was investigated.

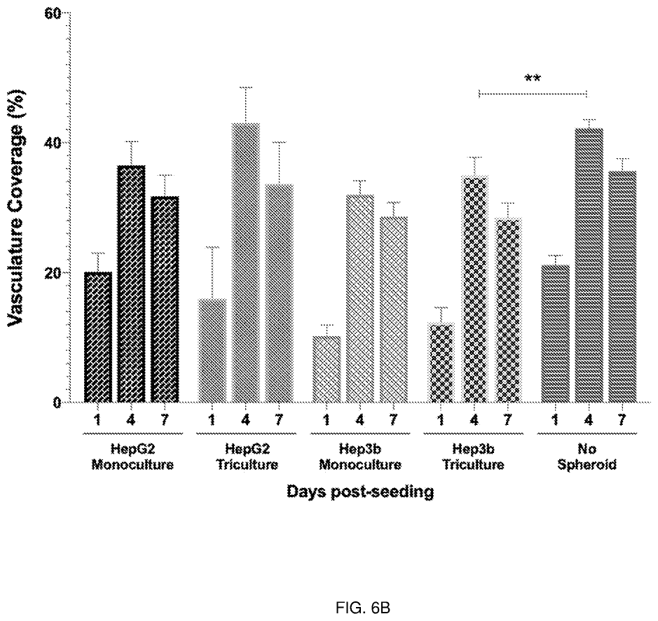

In this example, HepG2 and Hep3b liver tumour cell lines were used to form monoculture (only liver cancer cells) or triculture spheroids (liver cancer cell co-cultured with stellate cells (LX2) and endothelial cells). After 5 days of allowing the cells to organise and grow in an aggregate format outside the device, the spheroids formed were seeded into devices in an ECM containing GFP-HUVEC and NHLF. As a control, GFP-HUVEC and NHLF were seeded into the devices to monitor the natural formation of vessels. Representative images of the different conditions are shown in FIG. 6A.

Based on the data shown in FIG. 6A, vasculature quantification was performed using Fiji and the results are shown in FIG. 6B. For vasculature quantification, GFP-signal was quantified in the device excluding the spheroid and represented as a percentage of the total surface.

Effect of the Vasculature on Spheroid Viability

The effect of vasculature on spheroids cultured on the devices was also investigated.

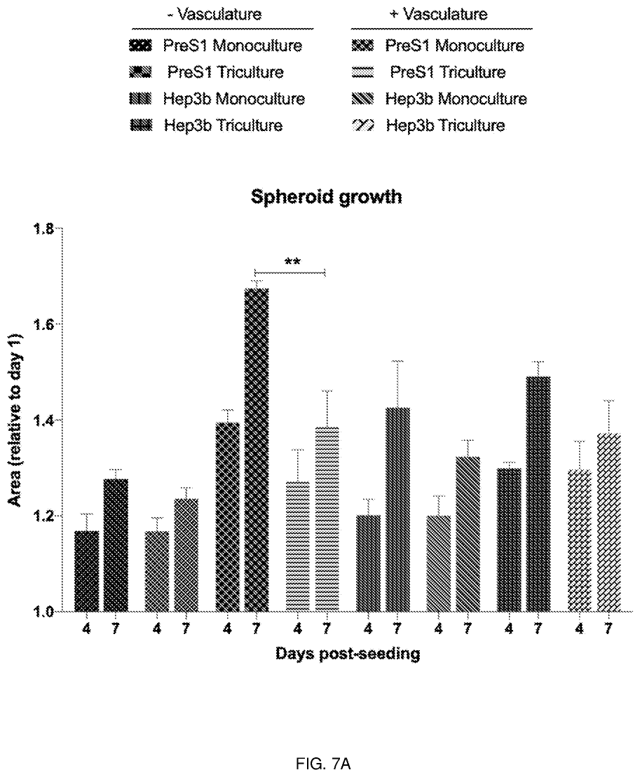

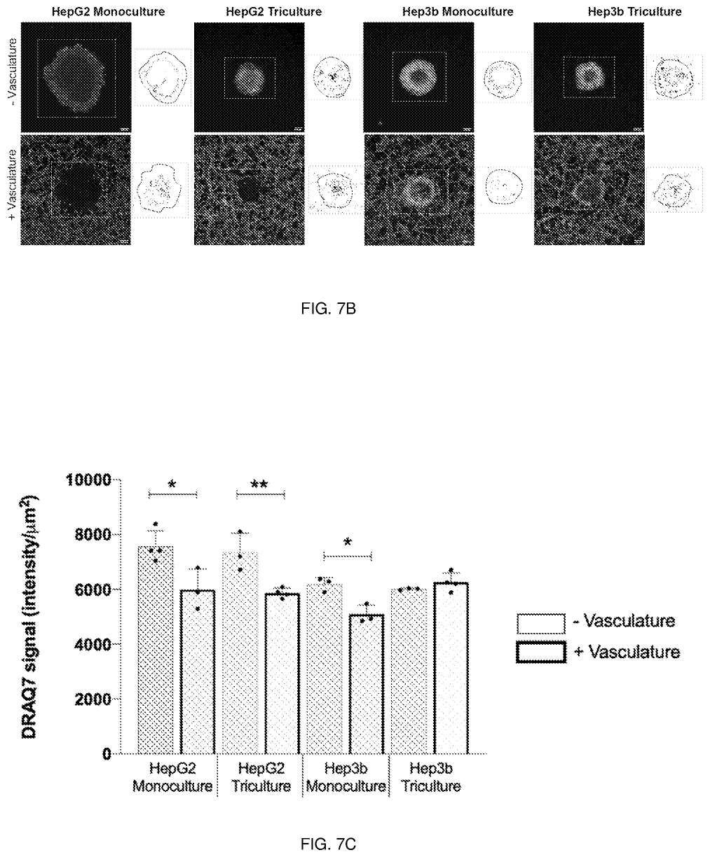

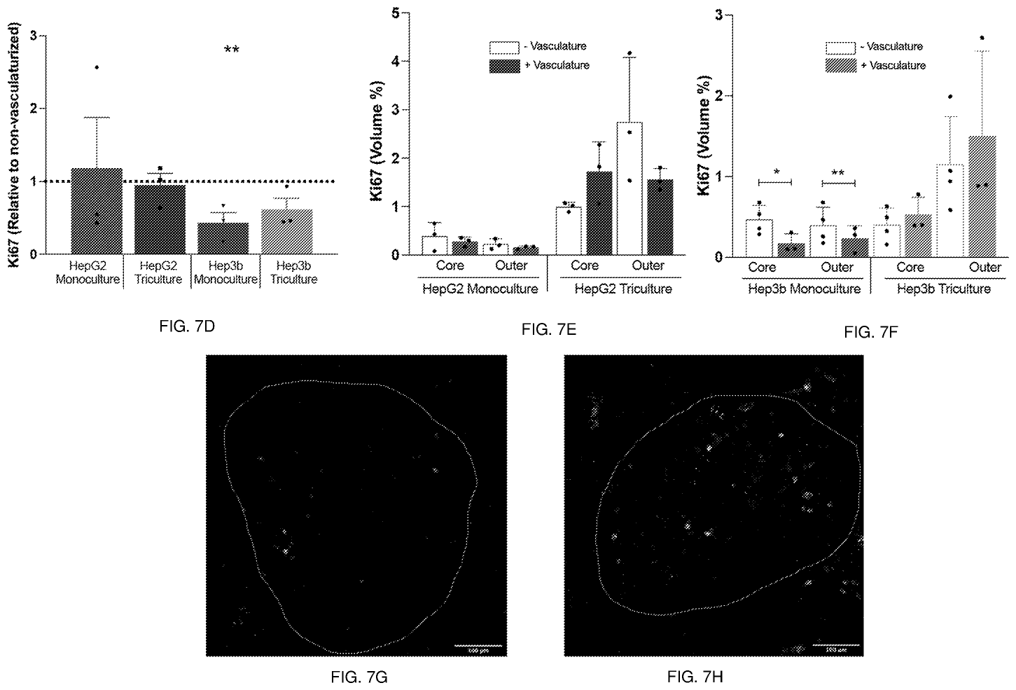

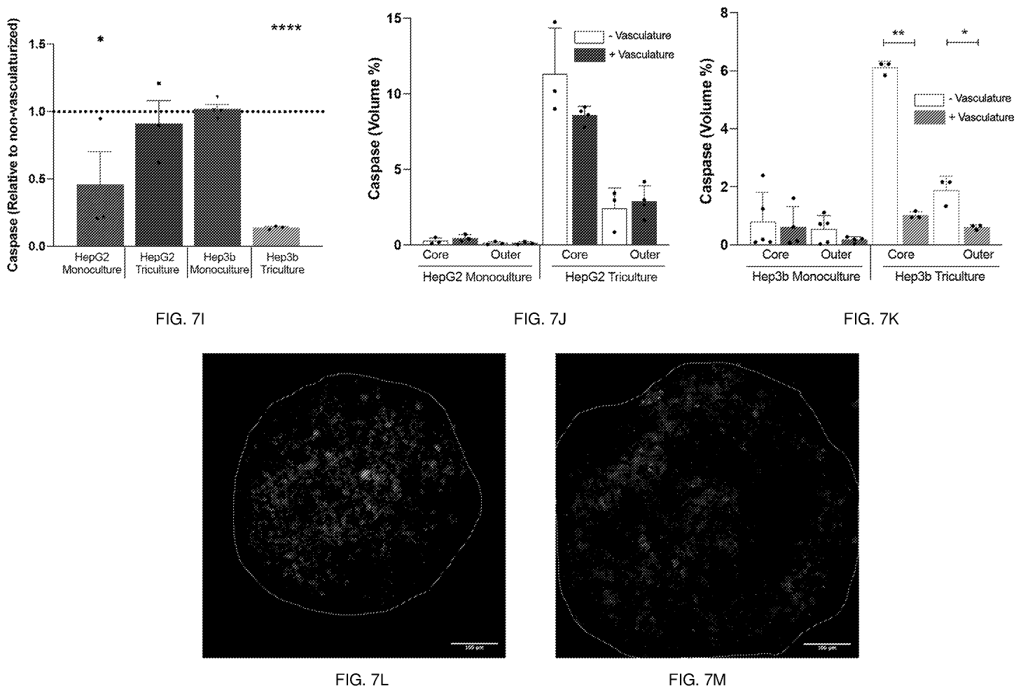

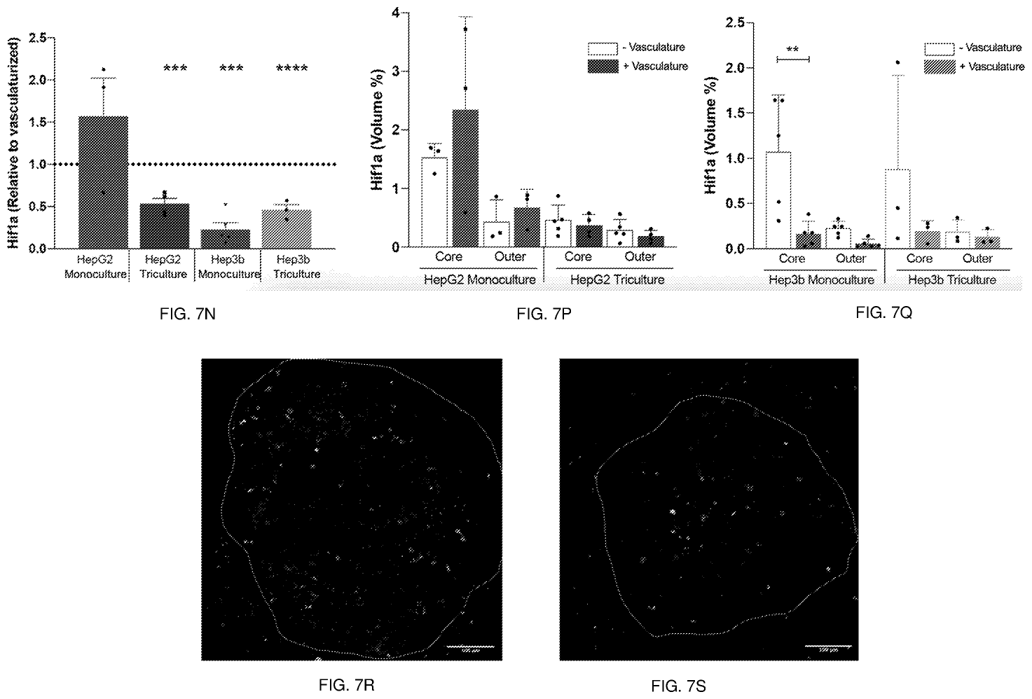

First, the effect of vasculature on spheroid growth was investigated. FIG. 7A is a graph showing spheroid growth over time measured with and without vasculature. As shown in FIG. 7A, the various spheroids showed growth with time (relative to day 1), both with and without vasculature.

Next, the effect of vasculature on the spheroid viability was quantified with DRAQ7. FIG. 7B shows representative fluorescence images, as well as expanded images of the boxed portions of the respective fluorescence images, from spheroids and DRAQ7 quantification. FIG. 7C is a graph showing quantification of DRAQ7 in the spheroids. As shown in FIG. 7C, spheroid viability was significantly improved when grown with vasculature, particularly for HepG2 monoculture spheroids, HepG2 triculture spheroids, and Hep3b monoculture spheroids.

The effect of vasculature on the expression of Ki-67 was also investigated. FIGS. 7D, 7E, 7F, 7G, and 7H show the distribution and expression of the proliferation marker Ki-67 in vascularised spheroids detected by immunofluorescence and compared to the proliferation in the spheroids grown in ECM without vasculature. As shown in FIGS. 7D, 7E, and 7F, a significant difference in expression of the proliferation marker Ki-67 was found particularly in Hep3b monoculture spheroids grown with vasculature, both in its core and outer regions of the spheroids. Overall the data show a decreased Ki-67 expression in cell aggregates grown with vasculature.

The effect of vasculature on apoptosis was also investigated. FIGS. 7I, 7J, 7K, 7L, and 7M show the extent of apoptosis in the spheroids detected by immunofluorescence using Caspase-3 antibody. As shown in these figures, vascularised spheroids mostly showed less apoptosis compared to spheroids in ECM without vasculature, suggesting a positive effect of the vasculature on the spheroids' viability. The difference was particularly significant for vascularised HepG2 monoculture spheroids (see FIG. 7I) and for vascularised Hep3b triculture spheroids, both in its core and outer regions of the spheroids (see FIGS. 7I and 7K).

The effect of vasculature on reducing hypoxia was also investigated. This was detected and measured using anti-Hif1 a (Hypoxia-inducible factor-1 alpha) antibody. As shown in FIGS. 7N, 7P, and 7Q (see also FIGS. 7R and 7S as examples), vasculature reduced hypoxia in most of the conditions tested. As shown in FIG. 7N, the presence of vasculature significantly reduced hypoxia, particularly for vascularised HepG2 triculture spheroids, vascularised Hep3b monoculture spheroids (especially in its core region, as shown in FIG. 7Q), and vascularised Hep3b triculture spheroids (also especially in its core region, as shown in FIG. 7Q).

Effect of Spheroid Type on Vasculature Formation

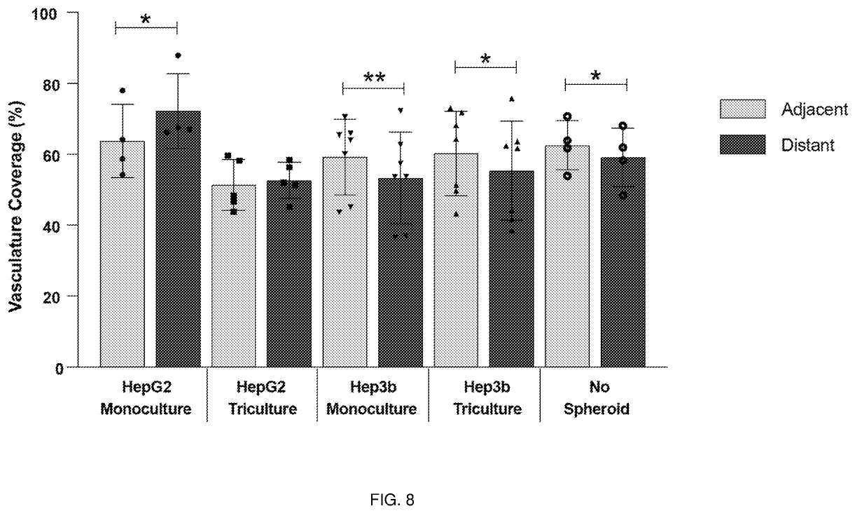

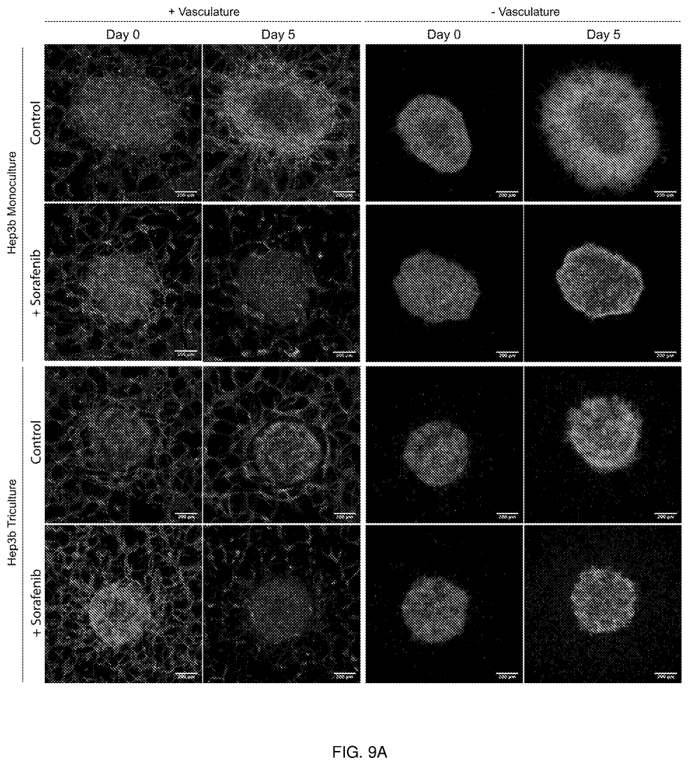

The effect of spheroid type on vasculature formation was investigated. For this, quantification of vasculature coverage close to the spheroid (or adjacent to the spheroid, wherein “adjacent” means within a distance of 350 μm from the spheroid) and distant from the spheroid (wherein “distant” means more than 350 μm from the spheroid) was performed using concentric analysis in Fiji and the results are shown in FIG. 8. As shown in FIG. 8, Hep3b spheroids were more angiogenic (vasculature coverage was close to the tumour) and monoculture HepG2 spheroids decreased the vasculature around the tumour compared to the region far/distant from the spheroid.

Effect of Drug Treatment on Tumour Spheroids and Vasculature

Next, the effect of Sorafenib treatment on the tumour spheroids and the vasculature was investigated. In this example, Hep3b monoculture and triculture spheroids were seeded in the devices with and without vasculature. At day 5, 10 μM of Sorafenib was used to treat the tumours for 7 days. GFP signal (representing the live cells) was quantified along the time.

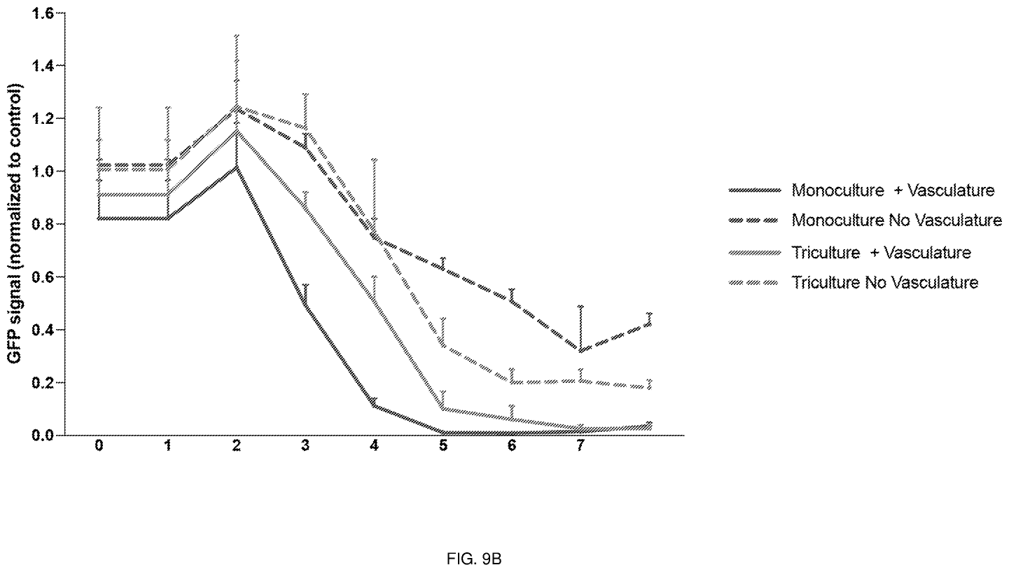

In FIG. 9A, representative images from all the conditions are shown at Day 0 (starting of the drug treatment) and Day 5 post-treatment. FIG. 9B is a graph showing the GFP signal over time represented from 3 independent replicates. As can be seen in the graph of FIG. 9B, Sorafenib transport is affected by vasculature. That is, in the Sorafenib treated samples the viability of the vascularised spheroids is lower compared to the non-vascularised spheroids, suggesting an improvement in drug delivery into the tumour due to the vasculature.

Use of Device to Test Cell Therapy

The use of the devices to test cell therapy was also investigated.

In the following first example, TCR-engineered T cells were used to test the efficiency in tumour killing of engineered T cells in 3D in vitro. HepG2 spheroids (TCR T cells targets) were seeded into the devices with and without vasculature. Vasculature was developed for 7 days. At day 7, 100 k TCR T cells or Mock T cells were added into the devices (to the side channels; compare fluidic regions 106 and 108 described with reference to FIG. 1C) and killing of the tumour was detected with DRAQ7 18 hours after the treatment.

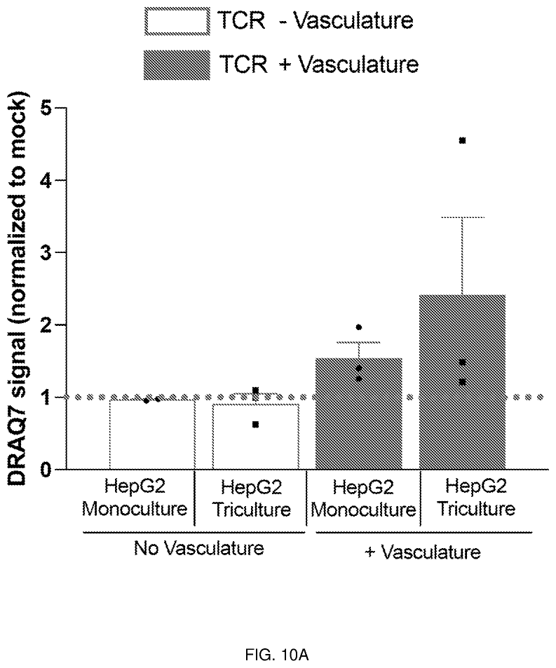

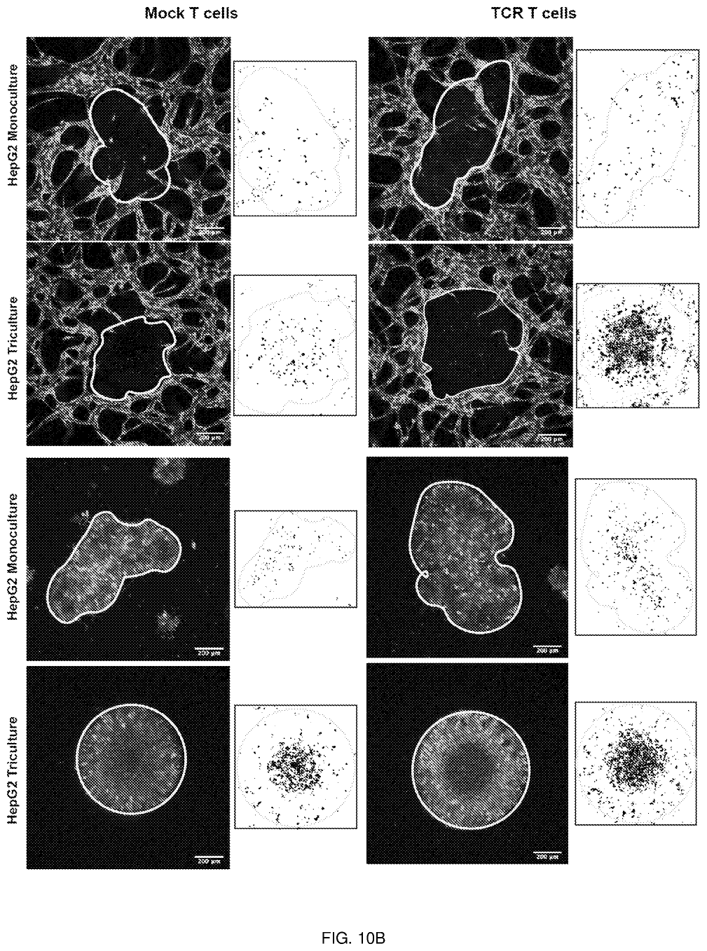

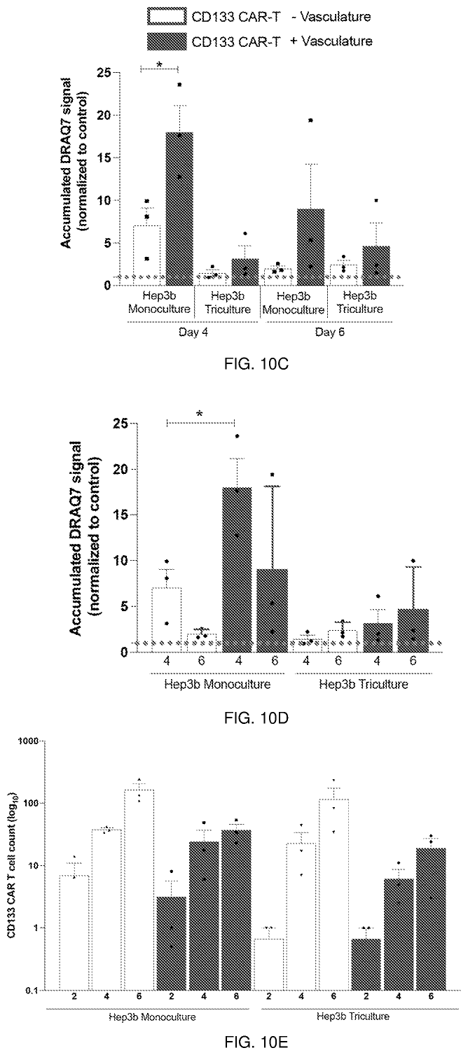

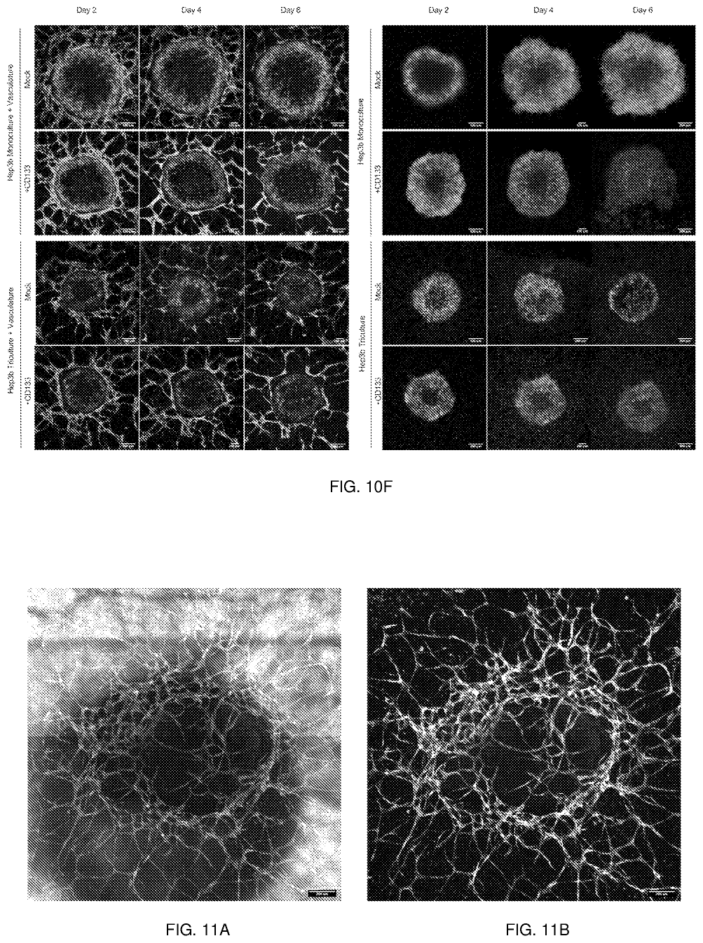

As shown in FIGS. 10A and 10B, TCR-engineered T cells showed an increase in killing HepG2 spheroids compared to Mock T cells in vascularised spheroids and no difference was detected in non-vascularised spheroids, suggesting than vascularisation of spheroids in the device can be used as a model to test cell therapies in vitro.

In the following second example, CD133 CAR-T cells were used instead to perform a killing assay in 3D in vitro in devices. Hep3b spheroids (CD133 CAR-T cells targets) were seeded into the devices with and without vasculature. 5 days post-seeding, 100 k CD133 CAR-T cells were added to the side channels of the devices and tumour killing was monitored using DRAQ7 at day 2, 4 and 6 post treatment and compared to the non-treated tumours (control).

As shown in FIGS. 10C and 10D, CD133 CAR T-cells were able to target and perform their cytotoxic activity on the spheroids in the devices in both vascularised and non-vascularised spheroids. For Hep3b monoculture spheroids in particular, a significant difference was observed at day 4 in the capability of the CD133 CAR T-cells to perform a higher cytotoxic activity on vascularised spheroids over the non-vascularised spheroids.

FIG. 10E shows that it's possible to quantify the amount of T cells reaching the tumor aggregate and that the CD133 CAR T-cell count grew from day 2 to day 6 in the devices with or without vasculature.

FIG. 10F shows representative fluorescence images of vascularised and non-vascularised Hep3b monoculture and triculture spheroids treated with CD133 CAR-T cells or mock cells.

Use of Device for Culturing Biopsies

The devices can be used to culture tumor biopsies. As an example, FIGS. 11A and 11B show images taken on day 3 of a vascularised patient biopsy (the biopsy was obtained from an ovarian cancer patient) which was cultured in a device with endothelial cells (HUVEC) and fibroblasts (NHLF).

### APPLICATIONS

Various embodiments of the devices and methods disclosed herein provide possible uses/applications in translational research, including drug screening and drug development; immunotherapy and cell therapy testing and QC (Quality Control); and personalised medicine strategies.

Various embodiments of the devices and methods disclosed herein also provide possible uses/applications in basic research, including: investigation of cancer development mechanisms, cancer vascularisation, and cancer metastasis; discovery of molecular pathways in cancer-endothelium-stroma interactions; and investigation on organoid differentiation and development.

Various embodiments of the devices and methods disclosed herein may be further utilised to develop an organ-on-a-chip platform that is ready for adoption in industry (i.e., develop from lab prototype version 1.0 to manufacturing-ready prototype version 2.0).

Various embodiments of the devices and methods disclosed herein may also be utilised to validate and optimise protocols for a human 3D complex vascularised cell aggregate in a device.

Various embodiments of the devices and methods disclosed herein may further be utilised to validate downstream analytics to analyse cellular spatial organisation in a 3D complex model, including histology characterisation.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A device for vascularising a cell aggregate, the device comprising:
a matrix region configured to contain a gel-like matrix and the matrix region having at least one opening for positioning the cell aggregate therein based on a desired three-dimensional spatial location; and
one or more fluidic regions configured to contain a supporting fluid that is capable of supporting vascularisation of the cell aggregate, the one or more fluidic regions being in fluid communication with the matrix region,
wherein a flow passage from the one or more fluidic regions to a gel-like matrix disposed in the matrix region is configured to allow three-dimensional vascularisation around the cell aggregate and perfusion of the vasculature once formed.

2. The device as claimed in claim 1, wherein one or more fluidic regions comprise at least two fluidic regions.

3. The device as claimed in claim 2, wherein one fluidic region is disposed lateral to the matrix region on one side and another fluidic region is disposed lateral to the matrix region on the opposite side.

4. The device as claimed in claim 1, wherein the flow passage is substantially free from intervening structural obstacles disposed between the one or more fluidic regions and the matrix region.

5. The device as claimed in claim 1, wherein the at least one opening is positioned substantially central to the matrix region.

6. The device as claimed in claim 1, wherein each fluidic region comprises at least two openings for facilitating introduction of the supporting fluid in each of the fluidic region.

7. The device as claimed in claim 1, wherein the matrix region is substantially symmetrical in shape along its longitudinal length.

8. The device as claimed in claim 2, wherein the at least two fluidic regions are symmetrically disposed about the matrix region.

9. The device as claimed in claim 1, further comprising a gel-like matrix disposed within the matrix region.

10. The device as claimed in claim 1, wherein at least part of walls defining the matrix region and the one or more fluidic regions comprises an elastomer.

11. The device as claimed in claim 1, wherein the device comprises an elastomer disposed on a substrate,
wherein the elastomer comprises patterns formed on an open surface of the elastomer, the patterns corresponding to a layout of the matrix region and the one or more fluidic regions, and
wherein the substrate substantially fluidically seals the patterns at the open surface of the elastomer to form the matrix region and the one or more fluidic regions of the device.

12. The device as claimed in claim 2, wherein the at least two fluidic regions are separated from one another by the matrix region.

13. A chip comprising a plurality of the device of claim 1.

14. A method for vascularising a cell aggregate, the method comprising:
providing a device comprising a matrix region configured to contain a gel-like matrix and the matrix region having at least one opening for positioning the cell aggregate therein based on a desired three-dimensional spatial location; and one or more fluidic regions configured to contain a supporting fluid that is capable of supporting vascularisation of the cell aggregate, the one or more fluidic regions being in fluid communication with the matrix region, wherein a flow passage from the one or more fluidic regions to a gel-like matrix disposed in the matrix region is configured to allow three-dimensional vascularisation around the cell aggregate and perfusion of the vasculature once formed;
introducing the gel-like matrix into the matrix region;
positioning a cell aggregate into the gel-like matrix via the at least one opening; and
introducing the supporting fluid into the one or more fluidic regions.

15. The method as claimed claim 14, wherein the gel-like matrix is substantially maintained in the matrix region by surface tension.

16. The method as claimed in claim 14, wherein the cell aggregate is positioned in the gel-like matrix such that the gel-like matrix fully surrounds the cell aggregate.

17. The method as claimed in claim 14, wherein the method further comprises introducing cells capable of supporting vascularisation of the cell aggregate into the one or more fluidic regions.

18. The method as claimed in claim 14, wherein the method further comprises introducing one or more cell types into the gel-like matrix capable of supporting vascularisation of the cell aggregate and/or interacting with the cell aggregate.

19. The method as claimed in claim 14, wherein the method further comprises vascularising the cell aggregate to obtain a three-dimensional vascularisation around the cell aggregate.

20. The method as claimed in claim 14, further comprising introducing one or more test agents into the one or more fluid regions and/or the matrix region; and
analysing the effect of the one or more test agents on the cell aggregate.
